(19) United States
(12) Patent Application Publication
(10) Pub. No.: US 2019/0380999 A1
(43) Pub. Date: Dec. 19, 2019

(54) COMBINATIONS OF GENISTEIN AND BISPHOSPHONATES

(71) Applicant: Primus Pharmaceuticals, Inc., Scottsdale, AZ (US)

(72) Inventor: Masayoshi Yamaguchi, Shizuoka City (JP)

(21) Appl. No.: 16/435,876

(22) Filed: Jun. 10, 2019

Related U.S. Application Data
(60) Provisional application No. 62/687,131, filed on Jun. 19, 2018.

(51) Int. Cl. A61K 31/353; A61K 31/663; A61P 19/10

(52) U.S. Cl. CPC A61K 31/353; A61P 19/10; A61K 31/663

(57) ABSTRACT

Methods of treating osteoporosis by administering synergistically effective amounts of genistein and a bisphosphonate, or pharmaceutically acceptable salts thereof.

### FIELD OF THE INVENTION

The present invention relates to methods of supporting and promoting bone health using combinations of genistein and bisphosphonates, and pharmaceutical formulations thereof.

### BACKGROUND OF THE INVENTION

Bone homeostasis is regulated through the functions of osteoclasts and osteoblasts (1-3). Bone is a dynamic tissue constantly remodeled by the sequential removal (bone resorption) of mature tissue by osteoclasts and its replacement (bone formation) through the deposition of newly formed mineralized matrix by osteoblasts (1-3). Osteoclasts are derived from hematopoietic progenitor cells and osteoblasts develop from bone marrow mesenchymal stem cells (1,2). Bone homeostasis is maintained through the actions of various hormones, cytokines and bone marrow environmental systems (1-3). Disturbance of this homeostasis induces bone loss (1). Aging leads to osteoporosis associated with a deterioration of bone mass through suppressed bone formation and enhanced bone resorption (4,5). Osteoporosis is widely recognized as a major public health problem (4,5). A notable manifestation of this disease is fracture of the proximal femur, the incidence of which increases as the population ages (4,5). Decreased bone mass in females is primarily due to reduced secretion of estrogen following the beginning of the menopause (5). Osteoporosis is an important cause of morbidity and mortality in elderly women. Development of a new supplemental strategy will be useful in the prevention and treatment of osteoporosis.

Bisphosphonates are a group of drugs that have a structural similarity to pyrophosphate, a high affinity for mineralized tissue, and were developed as agents for inhibiting osteoclastic bone resorption (6,7). These drugs have often been used as the first treatment option for osteoporosis since 1960s, when the first bisphosphonates were developed as drugs for human use (6,7). There are two main classes of bisphosphonates which differ in potency and mode of action, namely the low potency, non-nitrogen-containing bisphosphonates including coronate and ternate, and the more commonly used higher potency, nitrogen-containing bisphosphonates including alendronate, risedronate, ibandronate and zoledronate (6,7). Bisphosphonates are widely used for their multimodal bone-sparing action to prevent and treat osteoporosis in postmenopausal women, bone pain and hypercalcemia of malignancy (6,8,9). Oral bisphosphonates are used beneficially to reduce the risk of skeletal fractures in patients with osteoporosis and in cancer metastatic to bone (10). However, recent studies have suggested that the inhibitory effects of bisphosphonates on osteoclasts lead to impaired bone remodeling, bisphosphonate-related osteonecrosis of the jaw, gastrointestinal side effects and risk of cancer (11-13).

Botanical isoflavones, including daidzin, daidzein, genistein and genistein, are contained at relatively high concentrations in soybeans and other plants (14,15). Daidzin and genistin are hydrolyzed to daidzein and genistein, respectively, by β-glucosidase in the gastrointestinal tract (14,15). Among isoflavones, genistein has been demonstrated to have potent direct anabolic effects on bone metabolism in vitro (14-20), suggesting a role in the prevention of osteoporosis. Therefore, it is hypothesized that genistein may be useful in the prevention and treatment of osteoporosis.

### SUMMARY OF THE INVENTION

It has unexpectedly been discovered that the combination of bisphonates such as alendronate and genistein are able to synergistically suppress the proliferation and stimulate the death of preosteoclastic RAW264.7 murine monocytic cells in vitro. Moreover, the combination of alendronate and genistein was found to synergistically suppress the osteoclastic differentiation enhanced by RANKL, a known stimulator of osteoclastic differentiation in RAW264.7 cells. Alendronate and genistein when used separately did not show significant effects on the proliferation and death of RAW267.4 cells in vitro. However, the combination of the two agents acted synergistically to suppress the proliferation and increase the death of RAW267.4 cells, and subsequently suppress the differentiation of preosteoclastic RAW267.4 cells to mature osteoclasts. Furthermore, this combination exhibited a synergistic suppressive effect on RANKL-induced osteoclastic differentiation as compared with the effect of each agent alone. This suppressive effect on osteoclastic differentiation may be partly based on the proliferation-inhibiting and death-inducing effects of the combination on RAW267.4 cells, which decreased the number of preosteoclastic cells.

Thus, in a first principal embodiment, the invention provides (a) method of treating osteoporosis in a subject in need thereof comprising administering to said subject: (a) a therapeutically effective amount of genistein or a pharmaceutically acceptable salt thereof; and (b) a therapeutically effective amount of a bisphosphonate or a pharmaceutically acceptable salt thereof.

In a second principal embodiment the invention provides a method of suppressing osteoclastogenesis in a subject in need thereof comprising administering to said subject: (a) a therapeutically effective amount of genistein or a pharmaceutically acceptable salt thereof; and (b) a therapeutically effective amount of a bisphosphonate or a pharmaceutically acceptable salt thereof.

Additional advantages of the invention are set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawings, which are incorporated in and constitute a part of this specification, illustrate several embodiments of the invention and together with the description serve to explain the principles of the invention.

FIG. 1 is a series of bar graphs illustrating the synergistic suppressive effect on the proliferation of RAW267.4 cells in vitro from a combination of alendronate and genistein. RAW267.4 cells were cultured for 3 days in Dulbecco's modification of Eagle's medium containing vehicle (0.1% ethanol), (A) alendronate, (B) genistein or (C) alendronate plus genistein. After culture, the number of attached cells on the dish was counted. Data are presented as the mean±standard deviation of two replicate wells per data set using different dishes and cell preparation. *P<0.001 vs. control group (grey bar) as determined by one-way analysis of variance and Tukey-Kramer post hoc test.

FIG. 2 is a series of bar graphs illustrating the synergistic effect on the death of RAW267.4 cells in vitro from a combination of alendronate and genistein. RAW267.4 cells were cultured for 3 days in Dulbecco's modification of Eagle's medium, and then the cells were additionally cultured for 2 days in the presence or absence of (A) alendronate, (B) genistein or alendronate plus genistein. After culture, the number of attached cells on the dish was counted. Data are presented as the mean±standard deviation of two replicate wells per data set using different dishes and cell preparation. *P<0.001 vs. control group (grey bar) as determined by one-way analysis of variance and Tukey-Kramer post hoc test.

FIG. 3 is a series of bar graphs illustrating the synergistic suppressive effect on the osteoclast differentiation of RAW267.4 cells in vitro from a combination of alendronate and genistein. Cells were cultured for 6 days with RANKL (30 ng/ml) pre-incubated for 10 min with crosslinking anti-poly-histidine antibody (2.5 μg/ml) in the presence or absence of vehicle (0.1% ethanol), (A) alendronate, (B) genistein or (C) alendronate plus genistein. After 6 days of culture, the cells were fixed and stained for TRAP. TRAP⁺ multinucleated cells (at least three nuclei) were quantified and averaged from six cultures with two replicate wells per data set using different dishes and cell preparation and are presented as the mean±standard deviation. *P<0.05 or #P<0.001 vs. control group (grey bar) as determined by one-way analysis of variance and Tukey-Kramer post hoc test.

### DETAILED DESCRIPTION OF THE INVENTION

The present invention may be understood more readily by reference to the following detailed description of preferred embodiments of the invention and the Examples included therein.

### Definitions and Use of Terms

As used in this specification and in the claims which follow, the singular forms “a,” “an” and “the” include plural referents unless the context clearly dictates otherwise. Thus, for example, reference to “an ingredient” includes mixtures of ingredients, reference to “a therapeutic agent” includes more than one therapeutic agent, and the like.

“Osteoporosis” refers to a medical condition related to as decrease of bone mass, where bones become increasingly brittle or fragile due to the loss of calcium or vitamin D. Osteoporosis occurs when the creation of new bone does not keep up with the removal of old bone. Osteoporosis-related fractures commonly occur in the hip, wrist, and spine.

“Therapeutically effective amount” means a dose or amount that produces a therapeutic response or desired effect. Therapeutically effective amount refers to the quantity or amount of a substance that is required to correct the manifestations of a particular deficiency.

“Osteoblast” means a cell that synthesizes bone. Osteoblasts arise from mesenchymal stem cells and function in groups in the process of bone formation upon maturation. Specifically, osteoblasts form the functional part of the bone known as the bone matrix, which consists of protein and mineral.

“Osteoclastogenesis” is the development of osteoclasts, a type of bone cell that breaks down bone tissue. Osteoclasts are important to the maintenance, repair, and remodeling of bones. Osteoclasts derive from mononuclear precursor cells of the monocyte-macrophage lineage.

“Osteoclastic bone resorption” refers to the process in which osteoclasts disassemble bone and digest composite proteins and minerals. In the case of osteoporosis, bone breaks down much faster than it is renewed.

“Genistein” is chemically named 4′,5,7-trihydroxyisoflavone, and has the following chemical structure:

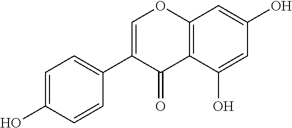 [SMILES: O=c1c(-c2ccc(O)cc2)coc2cc(O)cc(O)c12]

The compound is derived from natural soy and other botanical products in low amounts, and in its natural form, is conjugated to a glucoside group. In a preferred form for this invention, the molecule is present almost entirely as an unconjugated base, preferably having a purity of greater than 98 wt. %, and is referred to as genistein aglycone.

A dosage form, as used herein, refers to a formulation that is ready for administration to a subject. As used herein, it may refer to solid dosage forms, including, but not limited to, tablets, powders and capsules. Alternatively, it may refer to a liquid dosage form such as a solution or a suspension.

The term “about” or “ca.” accommodates industry standards for dietary supplements, medical foods and pharmaceuticals that allow for some level of variability within and among dosage forms and active ingredients. Thus, for example, a product that contains X mg of an active ingredient may contain a greater or lesser amount of ingredient X, to accommodate the imprecision associated with manufacturing processes, product degradation, and purity and potency variations in raw material supplies. In various embodiment, the term “about” accommodates variability in the active ingredient amounts of up to 10%, 5%, 3% or 2% (plus or minus).

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Therapeutically effective amount,” unless stated to the contrary herein, means that amount which, when administered to an animal for treating a disease or supporting a metabolic process, is sufficient to effect such treatment for the disease, or to support the metabolic process.

Weight percentages are given in terms of the element described. Therefore, a composition that contains 50 mg of alendronate or a pharmaceutically acceptable salt thereof would contain greater than 50 mg of the salt, but only 4 mg of molecular alendronate.

### DISCUSSION

In a first principal embodiment, the invention provides (a) method of treating osteoporosis in a subject in need thereof comprising administering to said subject: (a) a therapeutically effective amount of genistein or a pharmaceutically acceptable salt thereof; and (b) a therapeutically effective amount of a bisphosphonate or a pharmaceutically acceptable salt thereof.

In a second principal embodiment the invention provides a method of suppressing osteoclastogenesis in a subject in need thereof comprising administering to said subject: (a) a therapeutically effective amount of genistein or a pharmaceutically acceptable salt thereof; and (b) a therapeutically effective amount of a bisphosphonate or a pharmaceutically acceptable salt thereof.

Various subembodiments of the principal embodiments can also be described.

Thus, in one subembodiment of any of the foregoing principal embodiments, said therapeutically effective amount is effective to favor osteoblastic bone formation over the suppression of osteoclastogenesis.

In another subembodiment of any of the foregoing principal embodiments, said bisphosphonate is selected from the group consisting of alendronate, ibandronate, pamidronate, zoledronate, risedronate, etidronate, and pharmaceutically acceptable salts thereof.

In still another subembodiment of any of the foregoing principal embodiments, said therapeutic effective amount of genistein or pharmaceutically acceptable salt thereof comprises from about 25 to about 110 mg of genistein per day.

In even another subembodiment of any of the foregoing principal embodiments, said therapeutic effective amount of genistein or pharmaceutically acceptable salt thereof comprises about 54 mg of genistein per day.

In yet another subembodiment of any of the foregoing principal embodiments, said genistein and bisphosphonate, or pharmaceutically acceptable salts thereof, are administered in synergistically effective amounts.

### Pharmaceutical Compositions

Various pharmaceutical compositions can be developed that make use of the combinations described herein, but the composition is preferably administered orally in liquid or solid form. These compositions will generally include an inert diluent or an edible carrier. They may be enclosed in gelatin capsules (for oral use) or compressed into tablets (for oral or buccal use) or formulated into troches (for buccal use). For these purposes, the active compound can be incorporated with excipients and used in the form of tablets, troches, or capsules. Pharmaceutically compatible binding agents, and/or adjuvant materials can be included as part of the composition.

Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The active ingredient(s) can be administered as a component of an elixir, suspension, syrup, wafer, orally disintegrating film, orally disintegrating tablet, chewing gum or the like. A syrup may contain, in addition to the active compounds, sucrose as a sweetening agent and certain preservatives, dyes and colorings and flavors.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### Materials and Cells.

Dulbecco's Modification of Eagle's Medium (DMEM) with 4.5 g/l glucose, L-glutamine, sodium pyruvate and antibiotics [penicillin and streptomycin (P/S)] was purchased from Mediatech, Inc. (Corning, Manassas, Va., USA). Fetal bovine serum (FBS) was from Hyclone (GE Healthcare Life Sciences, Logan, Utah, USA). Alendronate, genistein, leukocyte acid phosphatase kits for tartrate resistant acid phosphatase (TRAP) staining and all other reagents were purchased from Sigma-Aldrich (Merck KGaA, Darmstadt, Germany) unless otherwise specified. The receptor activator of nuclear factor KB ligand (RANKL) was from R&D Systems, Inc. (Minneapolis, Minn., USA). Reagents were dissolved in 100% ethanol and sterilized distilled water. Mouse monocytic RAW267.4 cells were obtained from the American Type Culture Collection (Rockville, Md., USA) (21, 22).

### Cell Proliferation.

RAW267.4 cells (1×10⁵/ml cells/well; 2 ml medium added per well in 24-well plates) were cultured in DMEM containing 10% FBS and 1% P/S for 3 days in a water-saturated atmosphere containing 5% CO₂ and 95% air at 37° C. (23). The cells were cultured in the presence or absence of vehicle (ethanol; final concentration 0.1%), alendronate (0.1, 1, 10 or 100 μM), genistein (0.1, 1, 10 or 100 μM), or alendronate (0.1, 1, 10 or 100 μM) plus genistein (0.1, 1, 10, or 100 After culture, cells were detached from each culture dish and counted. Following trypsinization of each of culture dish using 0.2% trpysin plus 0.02% EDTA in Ca²⁺/Mg²⁺-free PBS for 2 min at 37° C., cells detached from the dish were collected following centrifugation at 150×g (4° C.) for 5 min (23). Cells were resuspended in PBS solution and stained with eosin. Cell numbers were counted under a microscope (Olympus MTV-3; Olympus Corporation, Tokyo, Japan) using a hemocytometer plate. For each dish, the average of two counts was calculated. Cell numbers are presented as the number per well.

### Cell Death.

RAW267.4 cells (1×10⁵/ml cells/well; 2 ml medium added per well in 24-well plates) were cultured in DMEM containing 10% FBS and 1% P/S for 3 days when confluence was reached (24). The cells were then cultured for an additional 2 days in the presence or absence of alendronate (0.1, 1, 10 or 100 μM), genistein (0.1, 1, 10 or 100 μM), or alendronate (0.1, 1, 10 or 100 μM) plus genistein (0.1, 1, 10 or 100 μM). After culture, cells were detached from each culture dish and counted as described for the cell proliferation assay.

### Osteoclastogenesis Assays and TRAP (Tartrate Resistant Acid Phosphatase, an Osteoclastic Marker) Staining.

RAW264.7 cells were cultured in 96-well plates in DMEM supplemented with 10% FBS and 1% P/S at a density of 1×10⁴ cells/well. Cells were cultured for 6 days with RANKL (30 ng/ml) pre-incubated for 10 min with crosslinking anti-poly-histidine antibody (2.5 μg/ml; IC050P; R&D Systems, Inc., Minneapolis, Minn., USA) to induce osteoclast formation (21,22), in the presence or absence of vehicle (ethanol; final concentration 0.1%), alendronate (0.1, 1, 10 or 100 μM), genistein (0.1, 1, 10 or 100 μM), or alendronate (0.1, 1, 10 or 100 μM) plus genistein (0.1, 1, 10 or 100 μM). After 6 days of culture, the cells were fixed and stained for TRAP, a specific marker of the osteoclast phenotype, using a leukocyte acid phosphatase kit. Briefly, cells were washed with PBS, fixed with 10% neutralized formalin-phosphate (pH 7.2) for 10 min, dried and then stained with leukocyte acid phosphatase kits for tartrate resistant acid phosphatase (TRAP) purchased from Sigma-Aldrich (Catalog No. 387A; Merck KGaA, Darmstadt, Germany) for 90 min at room temperature. TRAP-positive multinucleated cells (MNCs with at least three nuclei) were considered to be osteoclast-like cells, and the cells were counted using light microscopy (Olympus MTV-3). MNC scores are expressed as the mean±standard deviation of six cultures with two replicate wells per data set using different dishes and cell preparation.

### Statistical Analysis.

Statistical analysis was performed using GraphPad InStat software (version 3; GraphPad Software, Inc., La Jolla, Calif., USA). Multiple comparisons were performed by one-way analysis of variance, followed by a post hoc Tukey's range test for parametric data. P<0.05 was considered to indicate a statistically significant difference.

### Combination of Alendronate and Genistein Reveals a Synergistic Suppressive Effect on the Proliferation of RAW267.4 Cells.

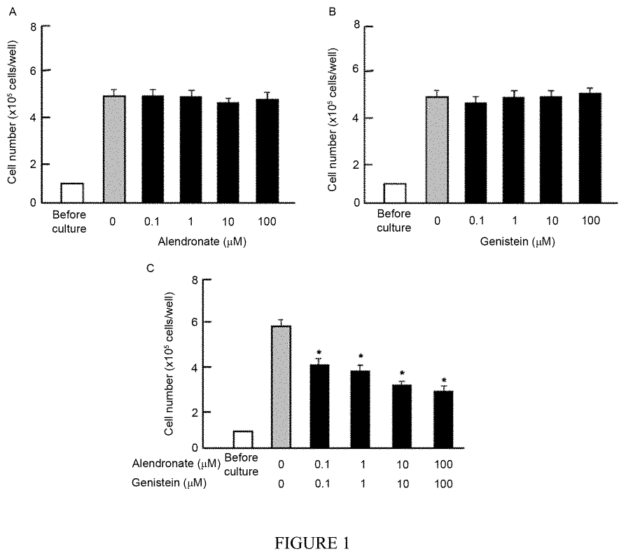

The effects of the bone metabolism regulators alendronate and genistein on the proliferation of RAW267.4 cells in vitro were examined. RAW267.4 cells were cultured for 3 days in the presence or absence of each compound. Culture with alendronate (0.1, 1.0, 10 and 100 μM) or genistein (0.1, 1.0, 10 and 100 μM) individually did not have a significant effect on the proliferation of RAW267.4 cells as compared with that of the control (0.1% ethanol vehicle; FIGS. 1A and B). Next, the effects of various combinations of alendronate and genistein on the proliferation of RAW267.4 cells in vitro were determined. Notably, combinations of alendronate and genistein with concentrations (0.1, 1.0, 10 and 100 μM) that did not independently reveal a significant effect on cell proliferation were found to synergistically suppress cell proliferation (FIG. 1C). Thus, the combination of alendronate and genistein was shown to possess a potent and synergistic suppressive effect on RAW267.4 cells in vitro.

Combination of Alendronate and Genistein Synergistically Stimulates the Death of RAW267.4 Cells In Vitro.

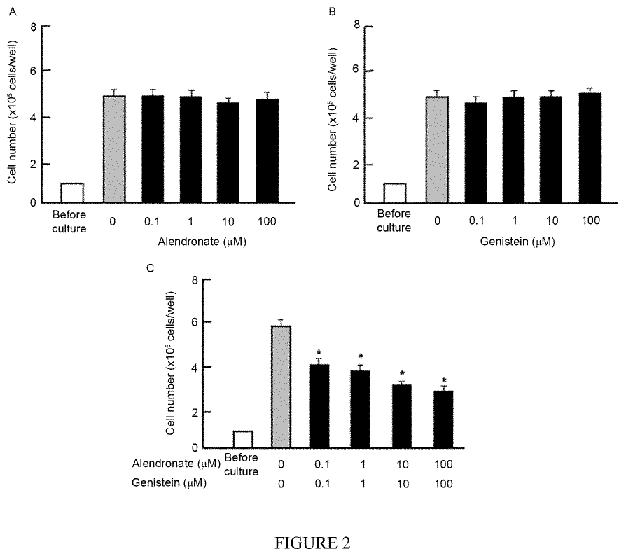

The effects of alendronate and genistein on the death of RAW267.4 cells in vitro were determined. Cells were cultured for 3 days until they reached confluence, and then the cells were additionally cultured for 2 days in the presence of 0.1% ethanol, alendronate (0.1, 1.0, 10 and 100 μM) or genistein (0.1, 1.0, 10 and 100 μM). Alendronate (0.1, 1.0, 10 and 100 μM) or genistein (0.1, 1.0, 10 and 100 μM) alone did not cause a significant alteration in the number of RAW267.4 cells (FIGS. 2A and B). However, combinations of alendronate (0.1, 1.0, 10 and 100 μM) and genistein (0.1, 1.0, 10 and 100 μM) significantly reduced the cell number, indicating that this combination treatment induces cell death (FIG. 2C).

### Combination of Alendronate and Genistein Synergistically Suppresses Osteoclast Differentiation of RAW267.4 Cells In Vitro.

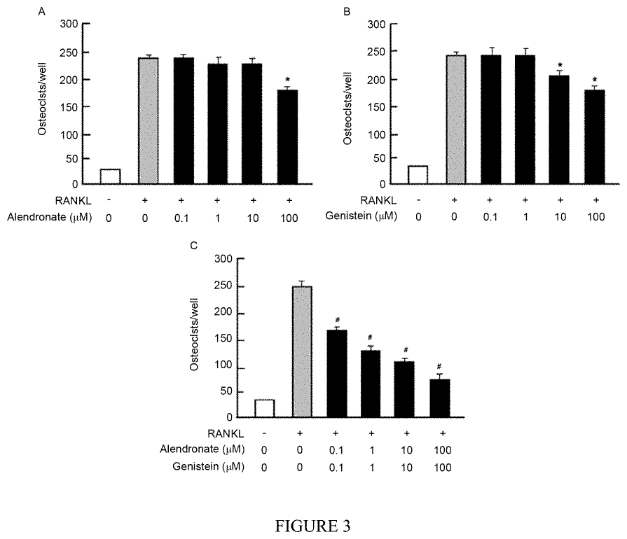

To establish an in vitro osteoclastogenesis model suitable for investigation of the activity of combined alendronate and genistein, RAW264.7 monocytic cells were induced to differentiate into osteoclasts by the addition of the key osteoclastogenic cytokine RANKL (2,3). The effects of alendronate and genistein on osteoclast differentiation were tested over a dose range from 0.1 to 100 μM; the cultures were stained with TRAP 6 days later and osteoclast formation was quantified. RANKL induced robust osteoclast formation (FIG. 3). Culture with alendronate (0.1, 1.0 and 10 μM) alone had no significant effect on the RANK-induced enhancement of osteoclastic differentiation (FIG. 3A). Genistein (0.1 and 1.0 μM) also did not reveal an effect on RANKL-stimulated osteoclastic differentiation. However, this stimulatory effect of RANKL was suppressed by addition of higher concentrations of alendronate (100 μM; FIG. 3A) and genistein (10 and 100 μM; FIG. 3B). Notably, combinations of alendronate (0.1, 1.0, 10 and 100 μM) and genistein (0.1, 1.0, 10 and 100 μM) were found to exhibit synergistic suppressive effects on the RANKL-induced enhancement of osteoclastogenesis in RAW267.4 cells in vitro (FIG. 3C).

### REFERENCES

1. Raggatt L J and Partridge N C: Cellular and molecular mechanisms of bone modeling. J Biol Chem 285:25103-25108, 2010.
2. Zaidi M, Blair H C, Moonga B S, Abe E and Huang C L-H: Osteoclastogenesis, bone resorption, and osteoclast-based therapeutics. J Bone Miner Res 18: 599-609, 2003.
3. Chambers T J and Fuller K. How are osteoclasts induced to resorb bone? Ann NY Acad Sci 1240:1-6, 2011.
4. Cooper C and Melton J III: Epidemiology of osteoporosis. Trends Endocrinol Metab 3: 224-229, 1995.
5. Weitzmann M N and Pacifici R: Estrogen deficiency and bone loss: an inflammatory tale. J Clin Invest 116: 1186-1194, 2006.
6. Fleisch H: Bisphosphonates: mechanism of action. Endocr Rev 19:80-100, 1998. Mottaghi P: Intravenous bisphosphonates for postmenopausal osteoporosis. J Res Med Sci 15:175-184, 2010.
8. Caro J J, Ishak K J, Huybrechts K F, Raggio G and Naujoks C: The impact of compliance with osteoporosis therapy on fracture rates in actual practice. Osteoporos Int 15:1003-1008, 2004.
9. Feng Z, Zeng S, Wang Y, Zheng Z and Chen Z: Bishosphonates for the prevention and treatment of osteoporosis in patients with rheumatic diseases: A systematic review and meta-analysis. PLos ONE 8:e80890, 2013.
10. Newcomb P A, Trentham-Dietz A and Hampton J M: Bisphosphonates for osteoporosis treatment are associated with reduced breast cancer risk. Br J Cancer 102:799-802, 2010.
11. Oh, Y H, Yoon C and Park S M: Bisphosphonate use and gastrointestinal tract cancer risk: Meta-analysis of observational studies. World J Gastroentero; 18:5779-5788, 2012.
12. Faiman B, Pillai L P C, Benghiac A G: Bisphosphonate-related osteonecrosis of the Jaw: Historical, ethical, and legal issues associated with prescribing. J Adv Pract Oncol 4:25-35, 2013.
13. Cardwell C R, Abnet C C, Veal P, Hughes C M, Cantwell M M and Murray L J: Exposure to oral bisphosphonates and risk of cancer. Int J Cancer 131:E717-E725, 2012.
14. Yamaguchi M and Gao-Balch Y H: Role of dietary soybean genistein in osteoporosis prevention. Int J Food Sci Nutr Diet 2:27-34, 2013.
15. Yamaguchi M: Nutritional factors and bone homeostasis: Synergistic effect with zinc and genistein in osteogenesis. Mol Cell Biochem 366:201-221, 2012.
16. Yamaguchi M and Gao Y H: Inhibitory effect of genistein on bone resorption in tissue culture. Biochem Pharmacol 55:71-76, 1998.
17. Gao Y H and Yamaguchi M: Inhibitory effect of genistein on osteoclast-like cell formation in mouse marrow cultures. Biochem Pharmacol 58: 767-772, 1999.
18. Gao Y H and Yamaguchi M: Anabolic effect of daizein on cortical bone in tissue culture: Comparison with genistein effect. Mol Cell Biochem 194: 93-98, 1999.
19. Yamaguchi M and Sugimoto E: Stimulatory effect of genistein and daizein on protein synthesis in osteoblastic MC3T3-E1 cells: Activation of aminoacyl-Trna synthetase. Mol Cell Biochem 214: 97-102, 2000.
20. Yamaguchi M and Weitzmann M N: The estrogen 17β-estradiol and phytoestrogen genistein mediate differential effects on osteoblastic NF-Kb activity. Int J Mol Med 23:297-301, 2009.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of treating osteoporosis in a subject in need thereof comprising administering to said subject:
a) a therapeutically effective amount of genistein or a pharmaceutically acceptable salt thereof; and
b) a therapeutically effective amount of a bisphosphonate or a pharmaceutically acceptable salt thereof.

2. A method of suppressing osteoclastogenesis in a subject in need thereof comprising administering to said subject:
a) a therapeutically effective amount of genistein or a pharmaceutically acceptable salt thereof; and
b) a therapeutically effective amount of a bisphosphonate or a pharmaceutically acceptable salt thereof.

3. The method of claim 1 or 2 wherein said therapeutically effective amount is effective to favor osteoblastic bone formation over the suppression of osteoclastogenesis.

4. The method of claim 1 or 2 wherein said bisphosphonate is selected from the group consisting of alendronate, ibandronate, pamidronate, zoledronate, risedronate, etidronate, and pharmaceutically acceptable salts thereof.

5. The method of claim 1 or 2 wherein said therapeutic effective amount of genistein or pharmaceutically acceptable salt thereof comprises from about 25 to about 110 mg of genistein per day.

6. The method of claim 1 or 2 wherein said therapeutic effective amount of genistein or pharmaceutically acceptable salt thereof comprises about 54 mg of genistein per day.

7. The method of claim 1 or 2 wherein the genistein and bisphosphonate, or pharmaceutically acceptable salts thereof, are administered in synergistically effective amounts.
